(19) United States
(12) Patent Application Publication
(10) Pub. No.: US 2009/0215034 A1
(43) Pub. Date: Aug. 27, 2009

(54) Method for selectively detecting subsets of nucleic acid molecules

(75) Inventors: Tamara Maes, Barcelona (ES); Richard Hampson, Lisbon (PT); Maria del Mar Benito Amengual, Barcelona (ES)

Correspondence Address: TRASKBRITT, P.C., P.O. BOX 2550, SALT LAKE CITY, UT, 84110, US

(73) Assignee: Oryzon Cenomics, S. A,, Barcelona (ES)

(21) Appl. No.: 11/578,001

(22) PCT Filed: Apr. 14, 2005
(86) PCT No.: PCT/EP2005/003787

§ 371 Date: Nov. 26, 2008

(30) Foreign Application Priority Data
Apr. 14, 2004 (ES) P 200400906

(51) Int. Cl. C12Q 1/68

(52) U.S. Cl. 435/6

(57) ABSTRACT

Method for selectively detecting nucleic acid molecules comprising structural aberrations that are capable of being convented into nicks comprising generating linear nucleic acids from a selected nucleic acid substrate population; denaturing and re-annealing the linear nucleic acids to form nucleic acid duplexes; masking the nucleic acid duplex termini and internal structural aberrations with a masking component; modifying the masked nucleic acids by introducing nicks therein using at least an enzyme possessing endonuclease activity: labelling the modified nucleic acids with labelled nucleotides via nucleic acid nick translation with at least an enzyme displaying a nucleic acid polymerase activity; and selecting and identifying the labelled nucleic acid.

The present invention relates to functional genomics and methods employed therein. In particular, there is provided a method for the detection of atypical structures, such as mutations or polymorphisms, in nucleic acids (NAs), and kits therefor.

### State of the Art

Recent technical advances such as industrial scale DNA sequencing have allowed characterisation of the entire sequence of a number of genomes from the simplest life forms to the most complex such as man. With this information insight is starting to be gained into, for example, evolution, genetic analysis of diseases and genetic identification. These techniques have undergone major development for use in fields such as pharmacology and preventive and forensic medicine.

A significant area of growth in the application of genome analysis techniques is in the diagnosis of disease, both hereditary and sporadic. Many diseases are caused by lost or altered gene function, often through changes in gene structure. Structural changes to a gene which can lead to an alteration therein, or loss of function thereto, range from a change or loss of a single nucleotide to the elimination of segments of deoxyribonucleic acid (DNA) which may be of millions of nucleotides in length.

Large changes are readily detected. A range of different techniques have been developed for the analysis of small scale changes to the genetic structure.

The numerous techniques for the detection of small scale mutations and polymorphisms fall into two groups, those for the detection of known mutations and those for the detection of unknown mutations.

It is possible to detect known mutations with high efficiency but there is scope for significant improvement in the number of individuals and the number of target sequences that can be analysed in one experiment.

Sensitive methods for the detection of known mutations include PCR (polymerase chain reaction) specific for one defined allele such as TaqMAMA [Glaab W. E., Skopek T. R. A novel assay for allelic discrimination that combines the 5′ fluorogenic nuclease polymerase chain reaction (TaqMan) and mismatch amplification mutation assay. Mut. Res. 430:1-12] and the detection of PNA (peptide nucleic acid) primer extension reactions by MALDI-TOF [Sun X., Hung K., Wu L. Sidransky D., B. Guo. Detection of tumour mutations in the presence of excess amounts of normal DNA. Nat. Biotech. 2002 February; 19:186-189].

It is of vital importance to distinguish methods designed to detect known mutations from methods which screen for new mutations.

Currently one of the most widely employed methods in the search of new mutations is direct sequencing, which makes use of dideoxynucleoside triphosphates for the termination of DNA synthesis.

Direct sequencing permits identification of changes in NA amplified with specific primers. Direct sequencing is however comparatively expensive, there is little scope for pooling of templates and there is a tight constraint on the length of DNA that can be analysed per reaction. Normally the limit per analysis lies between 300 and 600 base pairs (bp).

None the less, this method has been employed to great effect in generating a database of single nucleotide polymorphisms (SNPs) in the human genome, albeit at huge financial cost.

Other methods for screening for mutations are based on detection of DNA secondary structure and changes in DNA secondary structure as a function of sequence differences.

An example of such a technique is SSCP (single stranded conformational polymorphism) which takes advantage of the fact that heteroduplex and homoduplex NA molecules can be distinguished using polyacrylamide gels (with temperature or denaturant gradient) and HPLC analysis (high performance liquid chromatography) [McCallum et al, Targeted screening for induced mutations. Nat. Biotech. 2000 April; 18(4) :455-7].

Other methods for mutation screening have employed chemical agents and enzymes to recognize and process heteroduplex NA molecules and so aim to increase the capacity for discrimination.

Recognition of mismatches in heteroduplex NA molecules and atypical NA structures are routinely performed in two ways:

a) Chemically, for example the chemical detection of mismatches (regions of DNA where juxtaposed bases do not conform to Watson Crick base pairing rules) such as in the work by Cotton et al [Cotton R. G. H. et al. Reactivity of cytosine and thymine in single-base-pair base-mismatches with hydroxylamine and osmium tetroxide and its application to the study of mutations. (1988); Proc Natl Acad Sci USA, 85, 4397-4401].

b) Enzymatically, for example at sites of DNA damage [Harrison L. et al. (1999); In vitro repair of synthetic ionizing radiation-induced multiply damaged DNA sites. J Mol Biol, 290, 667-6841]and at sites of DNA mispairing [Oleykowski C. A et al. (1998); Mutation detection using a novel plant endonuclease NAR, 26, 4597-4602].

The processing of the heteroduplex or atypical DNA structures results in the cutting of one DNA strand (nicking) or of both DNA strands (cutting).

The analysis of fragments generated by such processing is easier than the direct distinction of homo and heteroduplex structures and can be performed e.g. by electrophoresis on sequencing gels [Oleykowski C A, Bronson Mullins C R, Godwin A K, Yeung A T. Nucleic Acids Res. 1998 October 15;26(20):4597-602; Colbert T, Till B J, Tompa R, Reynolds S, Steine M N, Yeung A T, McCallum C M, Comai L, Henikoff S. 2001 June; 126(2) :480-4].

Until the date of the present invention it is believed that most mutation screening methodologies are based on the direct detection of products resulting from the processing of heteroduplexes. This means that in virtually all cases mutant fragments are analysed in the presence of non-mutated fragments, both of which are present in the same relative proportions as in the original sample.

There are two notable exceptions which describe methods that combine mutation screening with high levels of detection sensitivity:

The first exception is the procedure described in U.S. Pat. No. 6,174,680. This method relies on the conversion of atypical DNA structures into abasic sites which are in turn covalently linked to a molecule which permits affinity purification. The level of detection of mutant molecules is 1% [Chakrabarti et al. (2000). Highly selective isolation of unknown mutations in diverse DNA fragments: toward new multiplex screening in cancer. Cancer Res. (60)3732-3737].

The second exception is a method based on the amplification of DNA fragments generated by heteroduplex processing and ligation of DNA adaptors described in US2003022215 and WO02/086169. The procedure described therein comprises the amplification of heteroduplex molecules after recognition and processing. To perform this procedure, heteroduplex DNA molecules with dephosphorylated 5′ termini are generated. Heteroduplex molecules are cut at the site of the mismatch, so revealing a new terminus which, in contrast to the pre-existing termini, is phosphorylated.

Synthetic adaptors are specifically ligated to these newly generated termini. Processed heteroduplex molecules can be distinguished by using a primer specific to the synthetic adaptor and a primer specific to the DNA fragment in a PCR reaction and obtaining an amplified product. Using this second method allows for the detection of mutants which represent 1% of the total mixture. [Zhang Y., Kaur M., Price B. D., Tetradis S., Makrigiorgos G. M., An amplification and ligation based method to scan for unknown mutations in DNA. Hum Mutat. 2002 August;20(2):139-47].

Rendering NA structures inert to the activity of certain enzymes has previously featured in other mutation detection methods.

The different methods described in the prior art to make NA structures inert are fundamentally different from the method described in the present invention.

US patent application US20030022215 (also WO02/086169), describes the ligation of an oligo/adaptor of DNA with dideoxynucleotides on the 3′ termini, in order to protect fragments from the pyrophosphorylation process carried out by DNA polymerase in the absence of free dNTPs (an enzymatic activity of DNA polymerase).

WO96/41002 teaches the possibility of blocking DNA ends by dephosphorylation to inhibit ligation, the addition of homopolymeric tails and ligation of modified double stranded DNA.

Nick translation is a classical molecular biology method which is employed as a general approach to labeling DNA and which has been further developed by Wong. Wong (US Patent application 20020187508) describes that nick translation may be used to label DNA molecules with a detectable group (by incorporation of a fluorescent group, a group that can be coupled to a fluorescent or radioactive entity etc.) and describes instruments which can be used to detect the molecules.

The method of US patent application 20020187508 cannot be applied to the procedure of the present invention since the enzymatic reactions employed therein are thought to work because the labelled molecule is being directly detected and not selected, which is a fundamentally different procedure to that described in the present application. In US patent application 20020187508 it is stated that DNA polymerase does not react with DNA termini.

This statement does not appear to correspond with the observations made and described in the present invention.

Referring to the blocking of DNA termini and damage to DNA molecules using ddGTP in a “DNA nick translation” reaction employing Taq DNA polymerase, a set of determined aspects have to be established which are delineated later in this document.

Previously described methods which constitute the state of the art for blocking DNA structures against enzymatic activity are not applicable to the present invention.

US20030022215 (also WO02/086169) employs ligation of a synthetic DNA fragment which contains a molecule that blocks activity of subsequently employed enzymes.

The use of DNA ligase is not compatible with the method of the present invention. In order for the present invention to be viable, it is necessary to block (mask) nucleic acid termini but also any internal damage within the nucleic acid molecules. Typically, such nucleic acid molecules comprise DNA.

WO96/4100 describes the use of heteroduplex molecules that are initially formed by hybridising a sample to be queried for mutations against a control sample affixed to a solid support, these are then cut and an adaptor joined to the fragments so generated. The fragments are then directly sequenced, employing an oligonucleotide primer specific to the adaptor.

In some aspects the protocol of WO96/4100 resembles that described by US20030022215 (also WO02/086169). Both protocols require the joining of an adaptor molecule to the site where the DNA has been cut in recognition of a heteroduplex region.

The difference between these two prior art methods stems from the additional blocking step that WO96/4100 employs in order to avoid joining of the adaptor to the original DNA termini which are present before cutting of the heteroduplex is performed. WO96/4100 also contemplates the adding of a homopolymeric deoxynucleotide tail and an initial ligation step with modified double stranded DNA.

US20030022215 (also WO02/086169) uses heteroduplex molecules which lack the 5′ phosphate group which are thus not templates to the ligation reaction.

In the procedure described by WO96/4100, after the ligation step, DNA is denatured and the fraction attached to the solid substrate eliminated. Remaining fragments are directly sequenced employing primers complementary to the adaptor and ligated to the heteroduplex molecule.

Finally, the sequencing reaction is performed employing standard dideoxynucleotide sequencing chemistry. However, employing this method, when either strand of reference DNA binds to the solid support in a non-selective manner for example, because the reference DNA has been amplified with biotinylated primers as described in WO96/4100, direct sequencing is not possible since the two strands will be read as an incoherent mixture.

Luchniak et al. [Biotech Histochem. 2002 January; 77(1):15-9] reports dideoxynucleoside triphosphates and Taq DNA polymerase are used to block in situ nick translation of undesired nicks in the DNA of whole chromosomes in plants (both whole cells and chromosome preparations).

Luchniak et al prefer Taq DNA polymerase over E. coli DNA polymerase I to perform this procedure as the 3′-5′ endonuclease activity associated with DNA polymerase I can eliminate the ddGTP incorporated in the blocking step.

In the work by Luchniak et al DNA termini are of no relevance. The idea is to block nicks generated in the DNA through the action of DNA degrading contaminants unavoidably associated with enzyme preparations employed for permeabilisation of cell walls.

In contrast, the present invention requires that nucleic acid termini, typically DNA termini, are of prime importance. The selection of Taq polymerase to supply enzyme activity is thus based on a fundamentally different rationale than that described by Luchniak et al.

For instance, relevant to the present invention, Taq DNA polymerase possesses, in addition to 5′-3′ DNA exonuclease and DNA polymerase activities, DNA terminaldeoxynucleotidyl transferase activity.

Thus, in the present invention dideoxynucleoside triphosphates may be employed to mask DNA ends and any pre-existing DNA damage from all the catalytic activities associated with Taq DNA polymerase during the labelling reaction.

In the method of Luchiak et al., protection and subsequent nick translation are performed at 62° C. In the examples of the present invention it is unequivocally shown that the reaction conditions described by Luchniak et al. are not functional in the method described herein.

For the recognition and processing of atypical structures to proceed in a desirable manner, multiple parameters should be defined in the method of the present invention as outlined herein. For example, recognition of mismatched sites in heteroduplex DNA may be carried out by Cel I nuclease (commercially available as SURVEYOR™) (or single strand specific nucleases such as mung bean nuclease and other members of the Si nuclease family) under favourable conditions, such as short incubation times as described herein.

United States Patents U.S. Pat. No. 6,391,557 and U.S. Pat. No. 5,869,245, describe a method for mutation detection based on Cel I (SURVEYOR™).

The enzyme Cel I (SURVEYOR™) can be used in the method of the invention but it is emphasized that the use of this enzyme is one example for a generic means of generating nicks in sites containing mismatches.

In order for mismatch endonucleases to function in the context of the present invention they must generate a single stranded nick and the 3′ OH (hydroxide) DNA terminus generated must be perfectly matched with the complementary strand. If there is a mismatch at the 3′ position, the Taq DNA polymerase exhibits a 100 to 1000000 fold reduced polymerase activity [Huang M M et al., Extension of base mispairs by Taq DNA polymerase: implications for single nucleotide discrimination in PCR. Nucleic Acids Res. 1992 September 11;20(17) :4567-73.].

When Cel I/SURVEYOR™ is applied to generate nicks in mismatch containing DNA in the procedure disclosed herein an activity which has previously not been attributed to this enzyme is employed [Oleykowski C A, Bronson Mullins C R, Godwin A K, Yeung A T. Nucleic Acids Res. 1998 October 15;26(20):4597-602]. There are two formal possibilities: either there is a significant endonuclease activity 5′ to the mismatch (in addition to that 3′ which has been described) or there is nicking 3′ to the mismatch followed by a nuclease activity which removes (at least) the mismatched site.

If there is only 3′ nicking activity the structure would not constitute a suitable substrate for subsequent Taq DNA polymerase catalysed labelling.

The agents employed in generating DNA nicks are not per se the subject of the present invention. In the procedure described in WO 97/46701, (also U.S. Pat. No. 5,869,245) it is claimed that the specificity of the mismatch recognition activity by the Cel I enzyme can be increased by its use in conjunction with other enzymes such as DNA ligase, DNA polymerase, DNA helicase, 3′-5′ DNA exonuclease and proteins which bind DNA termini, or a combination of such enzymes.

For this reason, the inventors emphasise the differences between the procedure described in WO 97/46701 (also U.S. Pat. No. 5,869,245) and the DNA protection procedure prior to the generation of double stranded DNA with nicks, as is described in the present invention.

WO 97/46701 presents as an example that if Taq DNA polymerase is added to a reaction of Cel I then Cel I specificity is elevated. The claims specifically state that the aim of adding the additional enzyme is to reduce non-specific action or increase turnover of the nicking reaction performed by the Cel I enzyme.

An object of the present invention is to provide a highly sensitive method that combines the capacity for searching for unknown mutations with increased sensitivity for detecting such mutations compared to methods known to date.

A further object of the present invention is to provide a method that permits specific labelling and recovery of molecules that contain atypical structures (such as non-Watson Crick base pairing). Thus, the detection of atypical DNA structures can be performed with significantly greater sensitivity than any other screening method to date.

A still further object of the present invention is to provide a sensitive procedure for the detection of any type of atypical NA structure that may be converted to a single strand cut (nick).

These and other objects of the invention will become apparent from the following description and examples.

### DESCRIPTION OF THE INVENTION

Atypical structures which can be converted to nicks are typically heteroduplex NA and any type of damage sustained by the NA. The procedure developed in the present invention provides an improved approach for the detecting of atypical NA structures, including heteroduplexes.

The method of the instant invention relies on masking undesirable reactive sites on the NA by incorporating blocking groups which render such sites non-reactive in a subsequent labelling step, thus contributing to the specificity of the method.

The method of the invention represents an inventive improvement on the methods described in the prior art. Advantages over methods of the prior art include extending the detection limit in a population to below 1%, and providing the capacity for identifying any type of atypical NA structure which can be converted to a nick. Such advantages are described in detail herein.

The present invention improves on the methods described in US Patents U.S. Pat. No. 6,391,557 and U.S. Pat. No. 5,869,245.

According to the present invention there is provided a process for selectively detecting nucleic acid molecules comprising structural features that are capable of being converted into nicks comprising:

a) generating linear nucleic acids from a selected nucleic acid substrate population;

b) de-naturing and re-annealing the linear nucleic acids to form nucleic acid duplexes;

c) masking the nucleic acid duplex termini and internal structural features with a masking component;

d) modifying the masked nucleic acids by introducing nicks therein using at least an enzyme possessing endonuclease activity;

e) labelling the modified nucleic acids with labelled nucleotides via nucleic acid nick translation with at least an enzyme displaying a nucleic acid polymerase activity; and

f) selecting and identifying the labelled nucleic acid.

Thus, for step a), the preparation of a linear nucleic acid population from a nucleic acid substrate population, for example, linear DNA from a DNA substrate population may be generated by PCR, for example by using a high fidelity DNA polymerase such as Pfu DNA polymerase, followed by denaturation and then renaturation, forming homo and/or heteroduplex molecules using conventional procedures [Sambrook, J., Fritsch, E F, and Maniatis, T. (1989). Molecular Cloning: A Laboratory Manual. Cold Spring Harbor Laboratory Press]. The nucleic acid substrate population may be derived from or selected from or obtained from any nucleic acid source be that natural or synthetic and may be obtained from RNA, genomic DNA, synthetic nucleic acids, such as cDNA, peptidic nucleic acid sources, synthesized non-viral or viral RNA, native viral RNA, mitochondrial or plastidial nucleic acids and the like. Damaged DNA such as ancient DNA from any suitable source could also act as a template. It is to be understood that “RNA” and “DNA” refer to both natural and/or synthetic sources unless context demands otherwise. Thus the nucleic acid substrate population may be derived or obtained or sampled from eukaryotic sources such as mammalian, fungal, yeast or plant (higher and/or lower order plant) sources, viral sources or prokaryotic, ie bacterial sources. Substrate nucleic acid populations may be obtained by any conventional means such as from biopsy samples of healthy or dysfunctional tissue. Nucleic acid termini and internal aberrations in the nucleic acid duplexes are then masked or protected to avoid non specific labelling in subsequent steps. Masking may be achieved through enzymatic incorporation of nucleotides or nucleotide analogues which terminate the DNA chain (such as dideoxynucleoside triphosphates or azidothymidine) using a suitable enzyme as the masking component as herein defined. An alternative approach to enzymatic masking could be by any direct conventional chemical conversion that renders DNA termini and internal aberrations non-reactive in the subsequent labelling procedure.

Typical masking conditions include adding a dideoxynucleotide analogue such as ddGTP in a nick translation reaction with Taq DNA polymerase wherein the incubation period may lie in the range of from 30 minutes to 18 hours, preferably masking is performed in the range of from 45 minutes to 10 hours, more preferably 60 to 120 minutes. The temperature at which the masking step is employed may lie between the range of from 37° C. to 60° C., preferably from 45° C. to 55° C., more preferably between 48° C. and 52° C.

Once masking of the nucleic acid duplex end termini and/or of any internal structural aberrations has been achieved, the masked nucleic acid molecules are modified by introducing nicks therein using at least an enzyme possessing endonuclease activity, such as Cel I “SURVEYOR™”, nucleases of the Cel family of mismatch endonucleases, mung bean nuclease, S1 nuclease or other single strand specific endoucleases [Till B J et al., Mismatch cleavage by single-strand specific nucleases., Nucleic Acids Res. 2004 May 11;32(8):2632-41]. Preferably, the modification is effected over a short time interval, typically in the range of from 2 to 7 minutes, using low enzyme concentrations, such as 10% of the concentration required for cutting (0.1-TILLING units [as defined in Till B J et al., Mismatch cleavage by single-strand specific nucleases., Nucleic Acids Res. 2004 May 11;32(8) :2632-41]).

Once the masked nucleic acid molecules have been modified, they may be labelled with labelled nucleotides via nucleic acid nick translation, typically using a nucleic acid polymerase such as E. Coli DNA polymerase I or Taq DNA polymerase. Labelling of the modified nucleic acid molecules, such as DNA molecules, whether native or synthetic in origin, is used to distinguish between nucleic acid molecules that have been modified as outlined herein from those that have not. Labelled nucleic acid molecules are then selected, for example using magnetic beads or particles covered with streptavidin and identified, for example by way of PCR amplification using conventional procedures [Sambrook, J., Fritsch, E F, and Maniatis, T. (1989). Molecular Cloning: A Laboratory Manual. Cold Spring Harbor Laboratory Press]. The individual steps outlined hereinabove are known and represent components of mutation detection methodologies including single stranded conformational polymorphism (SSCP) and a range of other methods for the detection of mutations in nucleic acid molecules [M, Iwahana H, Kanazawa H, Hayashi K, Sekiya T. Detection of polymorphisms of human DNA by gel electrophoresis as single-strand conformation polymorphisms. Proc Natl Acad Sci USA. 1989 April; 86(8):2766-70], the TILLING mutation detection system [McCallum C M, Comai L, Greene E A, Henikoff S. Targeted screening for induced mutations. Nat Biotechnol. 2000 April; 18(4):455-7, Henikoff S, Comai L. Single-nucleotide mutations for plant functional genomics. Annu Rev Plant Physiol Plant Mol Biol. 2003 June; 54:375-401].

We have discovered an auxiliary enzyme activity of Cel I nuclease, as previously indicated. Cel I removes labeled nucleotides from the 3′ end of linear DNA, either due to specific cleavage at the junction between double and single stranded regions or due to 3′-5′ exonuclease activity. Presence of Cel I 3′-5′ exonuclease activity could permit the labelling reaction to be performed with an enzyme harbouring DNA polymerase activity only rather than require additional 5′-3′ exonuclease activity.

However, the combination and order of the steps that make up the method of the invention has a substantial advantage over the methods of the prior art in that before the labelling step, a masking of, or protection of, intrinsic nucleic acid aberration or damage and masking of, or protection of nucleic acid termini eg DNA termini, is performed. Such masking (protection) avoids the indiscriminate labelling of DNA in the labelling reaction, so permitting the specific labelling of reactive sites revealed through the recognition and modification of atypical DNA structures.

The procedure disclosed herein, in common with the majority of prior art techniques follows on with direct analysis of products obtained by gel electrophoresis, analysis on a capillary sequencing machine, or by dHPLC (denaturing high performance liquid chromatography).

However, there are many shortcomings of the current state of the art. For example, enzymes applied under standard reaction conditions display low specificity and DNA is damaged during in vitro synthesis and manipulation.

The combination of the method steps of the present invention is not described in the prior art and furthermore, neither are the reaction conditions under which the method may be carried out. As a consequence, the inventiveness of the method of the invention is founded on the sequence of the steps in the procedure. Furthermore, in the attaining of such a workable method care was taken to establish certain reaction conditions upon which the invention could be carried out. The establishment of such reaction conditions represented a further inventive improvement over prior art processes.

In the present invention various aspects were identified which substantially improved the basic procedure as described hereinabove. Such aspects, as outlined herein when taken into account render the process suitable for large scale analysis as is required in functional genomics.

The inventors have established that:

a) Any population of DNA molecules, including DNA fragments obtainable via amplification, invariably contains damaged molecules. Such DNA damage has to be molecularly masked from subsequent labelling steps by incorporating nucleotides or other compounds (dideoxynucleotides or nucleotide analogues such as azacytidine) which impair DNA labelling.

b) All linear DNA fragments are actively labelled in the labelling reaction due to the presence of termini. Thus DNA termini have to be molecularly masked from subsequent labelling steps by incorporation of nucleotides or other compounds which impair DNA labelling.

c) The conditions for the processing of mismatches by specific endonucleases have had to be strictly optimised to cause nicking of one DNA strand rather than the cutting of both.

d) It is known that E. coli DNA polymerase I has 3′-5′ and not just 5′-3′ DNA exonuclease activity. Nonetheless it is widely considered that blunt DNA termini are inert to the 3′-5′ exonuclease activity.

e) In contradiction to the above-mentioned widely held belief, the inventors have shown that E. coli DNA polymerase I is capable of incorporating nucleotides at the extreme ends of DNA molecules. This means that all linear DNA fragments will be labelled and not only molecules in which reactive sites have been revealed through the processing of atypical structures such as mismatches. Taq DNA polymerase possesses 3′ terminal deoxynucleotidyl activity in addition to 5′-3′ DNA exonuclease activity and 5′-3′ DNA polymerase activity. Thus DNA termini have to be masked to avoid labelling in subsequent steps.

f) It was observed that the use of Taq DNA polymerase at 72° C., its temperature of maximum activity, compromises the efficiency of masking. The exact reason for this is unknown, but is thought to involve partial DNA denaturation or thermal DNA damage.

It was therefore necessary to define a temperature interval in which maximal specificity of labelling is combined with sufficient yield. The temperature interval for the masking step of the present invention is defined as being in the range of from 37° C. to 60° C., preferably in the range between 45° C. and 55° C., more preferably between 48° C. and 52° C.

g) The process used to generate nicks in DNA, for example by the action of endonucleases, may have a degree of non specificity. This may yield sites which can be labelled, so making the reaction less specific. For example we have observed that the mismatch endonuclease Cel I (SURVEYOR™) has 3′-5′ exonuclease activity and so renders DNA termini reactive to later labeling steps. It has been observed that any manipulation of the DNA during the mutation detection process, especially vortexing and precipitation, inflicts damage on the DNA. This molecular damage may be the site of initiation of labelling in subsequent steps and all care must be taken to minimise damage not only in substrate preparation but in all steps prior to labelling.

h) The selection process is a single tube reaction. This means that there is a contamination risk which must be monitored by a DNA fragment (without mutations) which can be identified by PCR and distinguished from other fragments which are being screened for mutations.

Taking into account all the requirements mentioned above the scheme of the procedure as applied to the detection of mutations, comprises: preparing a substrate nucleic. acid population; generating linear DNA therefrom, for example by PCR employing a high fidelity DNA polymerase such as Pfu DNA polymerase; denaturing and re-annealing of DNA fragments to permit formation of duplex and heteroduplex molecules; blocking (masking) of DNA termini and internal DNA damage using, for example, ddGTP in a nick translation reaction with Taq DNA polymerase using an incubation time typically of from 30 minutes to 18 hours in duration and a temperature typically in the range of from 37 to 60° C.; recognising and processing of atypical DNA structures using conditions that favour processing of atypical DNA structures to a nick. Such processing conditions include short reaction times, typically in the range of from 2 to 7 minutes at a temperature of from about 37° C. to 45° C., preferably for about 5 minutes at 42° C. and use of low enzyme concentrations, such as 10% of the amount of enzyme required for cutting as described hereinbefore.

There are a series of advantages to the masking process as described in the present invention. Naturally, the skilled addressee will appreciate that the advantages of the present invention as applied to DNA will also apply to other nucleic acid molecules (NA). Thus, the present invention is by no means limited to DNA and is applicable to any type of nucleic acid:

a) The aim of the protection procedure is neither to improve specificity of nicking of the atypical NA structure nor to increase turnover of the Cel I enzyme (commercially known as SURVEYOR™) in the nicking reaction. The absolute requirement for the masking step stems from the need to avoid DNA damage (intrinsic to molecules in any DNA population) and DNA termini (present in any non circular DNA molecule) from becoming the foci of the subsequent labelling reaction.

b) The enzyme employed in the protection reaction cannot be any of DNA ligase, DNA helicase 3′-5′ DNA exonuclease or a protein which binds to DNA termini. The enzyme(s) must be a DNA polymerase(s) having substantially no detectable 3′-5′ DNA exonuclease activity as detectable by conventional publicly available procedures but has 5′-3′ exonuclease activity or a combination of enzymes which can perform this reaction.

It is essential to note that in the present invention masking is not merely performed by enzymatic treatment of the substrate with a range of enzymes. Rather, the crucial part of this step is that the enzyme incorporates a component, such as a nucleotide analogue, into any damaged DNA and DNA termini in a step preceding the introduction of nicks into the double stranded DNA at/near the sites of atypical DNA structure. This incorporated component then efficiently blocks the sites at which it has been incorporated from the labelling during the subsequent labelling reaction.

However, that is not to say that when using Cel I (SURVEYOR™) to generate nicks in heteroduplex DNA the protection methodology used in the invention to avoid labelling at undesired sites also improves enzyme performance.

c) The purification of the modified DNA is required to change buffer conditions for the subsequent steps. All purification of DNA inflicts damage which can be picked up in the subsequent labelling reaction. Standard buffer change procedures such as DNA precipitation inflict unacceptably high amounts of DNA damage.

d) The labelling of modified DNA, for example, by the incorporation of biotinylated nucleotides by DNA nick translation using Taq DNA polymerase, using precisely defined time and temperature conditions.

e) Selection and identification of the biotin labelled DNA, for example using magnetic beads or particles covered with streptavidin and subsequently detection by PCR.

f) In the entire procedure, steps a) to f), an internal control free of mutations is incorporated to monitor selectivity of the procedure.

The method of the invention as disclosed herein, that is, for selectively detecting nucleic acid molecules comprising structural aberrations that are capable of being converted into nicks, can be applied to any nucleic acid molecule, such as ribonucleic acid (RNA) molecules, deoxyribonucleic acids (DNA), molecules which are chemically distinct from any natural NA which interact with NAs in a manner similar to normal NAs (such as PNAs) and any combination of these molecules.

The source of NAs for the template and substrate population may be viral, prokaryotic, eukaryotic, plasmid NA or a combination of any of the above. The substrate NA population may be generated by extraction, or by way of in vitro NA amplification using any conventional means employed in the art or it may be synthesised using any conventional means employed in the art.

Atypical NA structures may be the result of heteroduplex molecules formed from sources which harbour variability in that molecule. This variation may be natural or induced by physical, chemical or biological means.

Atypical NA structures may also be the result of ill effects of physical, chemical or biological agents. They may also occur as a result of intracellular enzymatic activities that can result in conversion of atypical NA structures into single strand nicks.

The man skilled in the art will appreciate that any high fidelity polymerase may be used instead of the Pfu DNA polymerase which was used to generate linear substrate DNA by PCR used in the invention.

Similarly, the man skilled in the art will appreciate that any agent which recognises mismatches in heteroduplex DNA molecules and is capable of introducing a nick in the DNA molecule [Mung bean nuclease, Kowalski D. et al. (1976) Biochemistry 15: 4457-4463, venom phosphodiesterase Pritchard A E et al. J. Biol. Chem. 1977; 252: 8652-8659] may replace the Cel I “Surveyor™” mismatch endonuclease in the generation of molecules for subsequent labelling. One such example is the combination of the MutY mismatch glycosylase [Au K G et al. Escherichia coli mutY gene product is required for specific A-G - - - C. G mismatch correction Proc Natl Acad Sci USA. 1988 December; 85(23):9163-6] and human AP endonuclease [Shaper N L, Grossman L, Purification and properties of the human placental apurinic/apyrimidinic endonuclease Methods Enzymol. 1980;65(l):216-24] or any other combination of a suitable glycosylase and a suitable AP endonuclease.

In the present invention, the step of NA protection before washing may be performed on any type of double stranded NA molecule which harbours atypical NA structures to which specific treatments to break one NA strand can be applied. Typically, the double stranded NA or single strand NA is DNA. For example AlkA 3 methyladenine DNA glycosylase [P. Karran, T. Hjelmgren and T. Lindahl. Induction of a DNA glycosylase for N-methylated purines is part of the adaptive response to alkylating agents 1982 Nature 296:770-773] together with human AP endonuclease [Shaper N L, Grossman L, Purification and properties of the human placental apurinic/apyrimidinic endonuclease Methods Enzymol. 1980;65(1):216-24] or any other DNA damage specific DNA glycosylase in conjunction with an AP endonuclease.

Once it has been established that it is crucial to eliminate or mask pre-existing DNA damage using nucleotide analogues such as ddGTP (dideoxyguanosine triphosphate) before the subsequent labelling with biotin it becomes possible to evaluate any modified nucleotide or other components to block the labelling of undesired sites.

The inventors emphasise that the use of equivalent components must always be evaluated in the context of the entire reaction procedure. This means that if a novel component, of which the properties in the context of this procedure are not completely defined, is used in the procedure, the overall outcome of the assay may be negatively affected, even though the component apparently perfectly replaces or improves on a component previously employed.

It is thus not sufficient to evaluate the possibility of replacing one component for another merely in the step where the replacement is due to happen. Rather it must be verified that subsequent steps are not also affected. It may for example be that a putative masking component yields efficient masking but is removed (unmasked) in the subsequent step designed to recognise atypical DNA structures which precedes the labelling. Or, on the other hand, a potentially efficient blocking component may be poorly incorporated into NA molecules.

Additional compounds to ddGTP that may be used in the masking reaction include AZT (azidothimidine) [Copeland W C et al. Human DNA polymerases alpha and beta are able to incorporate anti-HIV deoxynucleotides into DNA, J Biol Chem. 1992 October 25;267(30):21459-64] or any other nucleotide analogue capable of terminating DNA synthesis once it has been incorporated into DNA [Lim S E, Copeland W C, Differential incorporation and removal of antiviral deoxynucleotides by human DNA polymerase gamma. J Biol Chem. 2001 June 29; 276(26):23616-23].

In the present invention there is also incorporated a step where DNA molecules cut into a single strand are labelled with biotinylated deoxynucleoside triphosphates in a Taq DNA polymerase catalysed reaction. This reaction is catalysed by the 5′-3′ DNA polymerase and 5′-3′ DNA exonuclease activities of Taq DNA polymerase.

The man skilled in the art will appreciate that any enzyme or group of enzymes which combine such activities without harbouring further activities which impair the reaction may be used in place of Taq DNA polymerase. If such enzymes or enzyme combinations are identified and put to use, they must previously be evaluated in the full process.

Enzymes and enzyme combinations that may be employed in the labelling reaction instead of Taq DNA polymerase exist. For example Pfu DNA polymerase mutants lacking proofreading exonuclease activity, or exonuclease deficient Klenow fragments of DNA I polymerase in combination with a 5′-3′ DNA exonuclease, such as Fen 1 nuclease. In the same way, biotin may be replaced by other molecules which permit, directly or indirectly, separation of fragments into which they have been incorporated from fragments where there has been no incorporation.

Methods for separation may be via magnetic separation of beads coupled to an incorporated ligand, affinity chromatography for the ligand incorporated, flow cytometry and other non-destructive means for separation of molecules.

The man skilled in the art will appreciate that performing an amplification after the specific selection step stems from the need to visualise selected fragments on an agarose gel. However, the selection of fragments may be verified directly by employing any sufficiently sensitive method including mass spectrometry, hybridisation on any type of support (filter, DNA chips, beads) and atomic force microscopy.

Taking into account the basis of the invention disclosed herein, focusing on the intermediate steps of masking/protection by addition of analogous components to the NA sites that avoid specifically the labelling of protected DNA sites, a novel application has been developed.

One novel application of the procedure presented herein is concerned with the identification of differences between strains of the same species such as strains of the same yeast, for example of Saccharomyces cerevisae.

The procedure disclosed in the present invention can be used to identify:

a) Variation or differences between individuals of the same species.

b) Variation or differences in NA between cells of the same individual to, for example, distinguish between cancerous and non-cancerous cells.

c) Mutations in genes of interest in individuals of the same species.

d) Mutations in genes of interest in cells of the same individual, for example to identify cancer cells mutated in a specific gene.

e) DNA damage in sequences of interest, for example to selectively detect DNA damage in sequences of interest.

f) DNA damage at the whole genome level.

Also embraced within the scope of the present invention are kits for performing the method of the invention. For instance a kit suitable for use in the method of the invention may comprise means for preparing a population of linearised nucleic acid molecule(s), masking agents, such as ddGTP, AZT and the like, chemical or enzymatic masking components, nicking enzymes comprising endonuclease activity, labelling agents such as biotin and the like, enzyme preparations comprising or displaying nucleic acid polymerase activity.

Also embraced within the ambit of the invention is use of the inventive process described herein for the selective detection of atypical NA structures which can be converted to nicks; use of the inventive process described herein for the selective detection of variation or differences between the nucleic acid sequences of different variants of the same species; use of the inventive process described herein for the selective detection of variation or differences between nucleic acid sequences of different cells of the same individual; use of the inventive process described herein for the selective detection of mutations in genes of interest within a nucleic acid population; use of the inventive process described herein for the selective detection of mutations in genes of interest in different cells of an individual; use of the inventive process described herein for the selective detection of DNA damage in sequences of interest; use of the inventive process described herein for the detection of DNA damage at a genomic level.

There now follow figures and examples illustrating the invention. It is to be understood that the figures and examples are not to be construed as limiting the invention in any way.

### BRIEF DESCRIPTION OF THE FIGURES

FIG. 1 shows labelling by DNA nick translation of purified plasmid DNA.

FIG. 2 shows that labelling of DNA with biotin nucleotides using Taq DNA polymerase works at 37° C. but is much more efficient at 50° C.

FIG. 3 shows the efficacy of DNA deoxynucleotidyl transferase in DNA labelling which provides an alternative labelling method completely independent of DNA nick translation.

FIG. 4 shows the undesirable interference of DNA ligase with DNA nick translation.

FIG. 5 illustrates the theoretical considerations as to how DNA exonuclease and DNA polymerase activities may lead to labeling of DNA termini.

The bold line indicates the outcome of DNA polymerase activity, i.e. indicating DNA labeling by the DNA polymerase.

FIG. 6 shows that masking of DNA damage by E. coli DNA polymerase I using ddGTP. drastically reduces the amount of labelling carried out.

FIG. 7 shows that E. coli DNA polymerase I indiscriminately labels the ends of linear DNA irrespective of whether the DNA has been masked with ddGTP or not.

FIG. 8 shows that masking DNA damage and DNA termini with ddGTP using Taq DNA polymerase minimises background noise in DNA nick translation of linear DNA.

FIG. 9 shows ddGTP masking using Taq DNA polymerase at different temperatures and that incorporation functions optimally at 50° C.

FIG. 10 shows detection of mismatches processed by mung bean nuclease.

FIG. 11 shows that mismatch recognition and modification with low levels of SURVEYORT™ nuclease and short incubation times permit efficient trapping of nicked DNA (processed heteroduplex DNA).

FIG. 12 shows the prejudicial effect of standard ethanol precipitation of DNA on masking.

FIG. 13 shows that spin column purification provokes little increase in background labelling.

FIG. 14 shows the result of an entire process of the invention and that the method is sufficiently sensitive to efficiently detect one mutant molecule in the presence of 255 normal molecules.

FIG. 15 shows the identification of variations between different strains of the same yeast species using the method of the invention.

FIG. 16 shows the use of the method of the invention in a SAMPAD screening experiment, efficiently identifying one mutant molecule per 128 molecules.

FIG. 17 shows the identification of mutations in the human adenomatous polyposis coli (APC) gene using the method of the invention.

### EXAMPLES SECTION

Experimental examples shown in the following section are described solely to support specific aspects of the present invention described herein.

In the following individual experiments are described that show how SAMPAD (selective detection and amplification of mutations, polymorphisms and DNA damage), is carried out according to the method of the present invention.

In Examples 1-8, different ways of performing individual components of SAMPAD are described.

In Examples 9-11, methods for labelling DNA are shown.

Various efficient methods to reduce background noise in the labelling reaction have been developed (Examples 12 to 17) and for combining the individual steps (Examples 18, to 21) to perform the entire SAMPAD process (Example 22). Example 23 illustrates technical possibilities and example 24 illustrates an industrial application of SAMPAD.

### Example 1

### Generation of a Wild Type Model Template

The template used was based on plasmid pUC18 digested at the unique NotI site (1 hour at 37° C. in the presence of 50 mM Tris-HCl (pH 7.5), 10 mM MgCl₂, 100 mM NaCl, 0.1 mg/ml BSA, 10 U NotI in a total volume of 20 μl. The product of the digestion was ligated to the annealed product of the two synthetic oligonucleotides identified as SEQ ID No.1 and SEQ ID No.2.

The DNA ligation reaction was carried out for 3 hours at 37° C. in the presence of 1 unit T4 DNA ligase, 40 mM Tris HCl, 100 mM MgCl₂, 10 mM DTT and 0.5 mM ATP.

Chemically transformation competent E. coli DH5α were transformed with the ligation product in a thermal shock procedure (30 minutes on wet ice). The shock treatment was followed by culture of the cells in non selective LB broth at 37° C. Subsequently transformants were selected for their resistance to antibiotics. The DNA substrate for SAMPAD was generated by PCR amplification with Pfu DNA polymerase.

### Example 2

### Generation of a Mutant Model Template

The template used was based on plasmid pUC18 digested at the unique NotI site (1 hour at 37° C. in the presence of 50 mM Tris-HCl (pH 7.5), 10 mM MgCl₂, 100 mM NaCl, 0.1 mg/ml BSA, 10 U NotI in a total volume of 20 μl. The product of the digestion was ligated to the annealed product of the two synthetic oligonucleotides identified as SEQ ID No.3 and SEQ ID No.4.

These oligonucleotides were derived from the nucleotides of Example 1 but now contained a TA insertion.

The DNA ligation reaction was carried out for 3 hours at 37° C. in the presence of 1 unit T4 DNA ligase, 40 mM Tris HCl, 100 mM MgCl₂, 10 mM DTT and 0.5 mM ATP.

Chemically transformation competent E. coli DH5α were transformed with the ligation product in a thermal shock procedure (30 minutes on wet ice). The shock treatment was followed by culture of the cells in non selective LB broth at 37° C. Subsequently transformants were selected for their resistance to antibiotics. The DNA substrate for SAMPAD was generated by PCR amplification with Pfu DNA polymerase.

### Example 3

### Substrate Preparation for SAMPAD

Model substrate DNA molecules were amplified to generate substrate DNA using Pfu polymerase under standard reaction conditions.

Standard conditions were as follows:
a) Reaction volume: 20 μl,
b) 200 pg of model substrate DNA,
c) 2 μl 10×Pfu DNA polymerase reaction buffer: 200 mM Tris-HCl (pH 8.8 a 25 oC); 100 mM (NH4)2SO4; 100 mM NaCl; 1% Triton×100 1 mg/ml bovine serum albumin and 20 mM MgSO4,
d) 5.12 μl dNTPs (12.5 μM each)
e) 0.5 μl of each primer (M13 sequencing primers SEQ ID No.5 and SEQ ID No.6, concentration 10 μM)
f) 0.5 U Pfu DNA polymerase.

Substrates were quantified by agarose gel electrophoresis, diluted as required and mixed in defined proportions (1:0 (=matched) to 1:256).

Mixtures were denatured by incubation at 95° C. and renatured by gradual cooling to room temperature over a period of at least 9 hours.

The resultant heteroduplex molecules were 1900 bp linear DNA molecules with two extrahelical bases at position 944.

Duplex molecules were identical with the exception of mismatched nucleotides.

### Example 4

### Masking of DNA Molecules with ddGTP

Masking reactions were carried out for 2 hours at a temperature of 50° C.

Reaction composition was as follows:
g) 2 μl DNA (8, 5 ng/μl),
h) 2 μl 10×Taq polymerase reaction buffer (160 mM (NH₄)₂SO₄; 670 mM Tris-HCl (pH 8.8 a 25° C.) and 0.1% Tween 20),
i) 0.75 μl 25 mM MgCl₂,
j) 5 μl ddGTP mix (2 μl 10 mM dATP; 2 μl 10 mM dCTP; 2 μl 10 mM dTTP; 2 μl 10 mM ddGTP and 72 μl distilled H₂O),
k) 10 U Taq DNA polymerase
l) 10 μl distilled H₂O (final volume 20 μl).

### Example 5

### Nicking Heteroduplex DNA with SURVEYOR™

SURVEYOR™ (Transgenomic, Omaha, Neb., USA) is the commercial name of an enzyme of the Cel I nuclease subfamily of S1 nucleases members of which were first obtained from celery.

The SURVEYOR™ reaction was performed in a volume of 20 μl. 8.5 ng DNA were incubated with 0.1 μl SURVEYOR™ and 2 μl 10× SURVEYOR™ reaction buffer for 5 minutes at 42° C.

### Example 6

### Labelling of DNA with Biotin

DNA (8.5 ng) was labelled by incorporation of biotin 11 dCTP. The reaction was performed in a volume of 30 μl with 3 μl 10× Taq DNA polymerase reaction buffer (160 mM (NH₄)₂SO₄, 670 mM Tris-HCl (pH 8.8 at 25° C.), 0.1% Tween 20); 0.75 μl 25 mM MgCl₂; 5 μl biotin dNTP mix (2 μl 10 mM dATP, 2 μl 10 mM dGTP, 2 μl 10 mM dTTP, 1.92 μl 10 mM dCTP y 0.8 μl biotina 11 dCTP) and 10 U Taq DNA polymerase.

Subsequently, biotin labelled fragments were selected by magnetic beads or particles coated with streptavidin. Streptavidin and biotin have very high binding affinity for each other, so permitting selection of biotin labelled molecules.

### Example 7

### Selection of Biotin Labelled DNA Molecules

Initially beads/particles were washed with twice their original volume of TEN 100 (10 mM Tris HCl, 1 mM EDTA 100 mM NaCl; pH 7.5). After each wash a magnet was applied and the supernatant removed.

Then beads/particles were resuspended in a volume of TEN 200 (10 mM Tris HCl, 1 mM EDTA and 200 mM NaCl; pH 7.5) equal to their inial volume.

Then 7.5 ng internal control DNA, 30 μl of the labelling reaction were added to 20 μl of washed beads.

In the fourth step of this procedure the mixture was incubated at room temperature for 30 minutes, repeatedly agitated to avoid settling of the beads/fragments. Then pre-wash samples for PCR were taken and the residual beads washed three times in each 400 μl TEN 1000 (10 mM Tris HCl, 1 mM EDTA and 1 M NaCl at pH 7.5). After each wash the magnet was applied to sequester the beads and the supernatant was removed. If more washing cycles were required this step was repeated accordingly. Finally, beads were resuspended in 40 μl dH₂O and samples taken for PCR.

### Assay 8

### Identification of Selected DNA

Identification was performed by PCR using the specific primers SEQ ID NO. 7 and SEQ ID NO.8.

Products were separated by agarose gel electrophoresis in 1×TAE buffer and visualised by ethidium bromide staining and UV transillumination.

### Example 9

### Development of a Method for Labelling, Selection and Identification of DNA

Plasmids were isolated from liquid bacterial cultures (2 ml of an overnight culture under selective conditions) using standard plasmid minipreparation techniques. Briefly, cells were collected by centrifugation and resuspended in resuspension buffer (50 mM glucose, 25 mM Tris HCl (pH=8) y 10 mM EDTA at pH 8), lysed by addition of a second solution (0.2 N NaOH and 1% SDS) and neutralised by addition of acetic acid to a final concentration of 11.5%.

Finally DNA was extracted with phenol chloroform and recovered by ethanol precipitation.

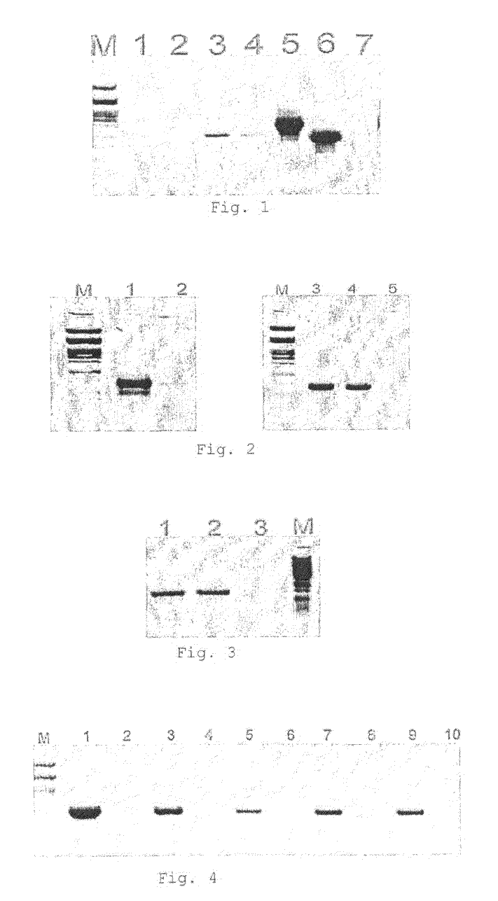

Approximately 200 ng of thus purified plasmid DNA was labelled as in Example 6 with the modification that E. coli DNA polymerase I and 10× E. coli DNA polymerase I reaction buffer were used instead of Taq DNA polymerase and 10× Taq DNA polymerase reaction buffer and the incubation accordingly performed at 37° C. for 30 minutes. In FIG. 1 we demonstrate that purified plasmid DNA is a good substrate for labelling by DNA nick translation. In FIG. 1 the lanes, from left to right are as follows:
a) M: λPst marker
b) 1: PCR of a mixture of plasmids A and B without biotin labelling, identified after 3 rounds of selection.
c) 2: PCR of a mixture of plasmids A and B without biotin labelling, identified after 6 rounds of selection.
d) 3: PCR of a mixture of plasmids A and B, where A but not B is labelled with biotin, identified after 3 rounds of selection.
e) 4: PCR of a mixture of plasmids A and B, where A but not B is labelled with biotin, identified after 6 rounds of selection
f) 5: positive control PCR of plasmid A.
g) 6: positive control PCR of plasmid B.

### Example 10

### Labelling of DNA with Biotin Nucleotides

In FIG. 2 we show that labelling of DNA with biotin nucleotides works at 37° C. but is much more efficient at 50° C.

The DNA template was prepared as in Example 1, the substrate was prepared as in Example 3.

DNA was labelled as in Example 6, selected as in assay 7 and identified as in Example 8.

The results of this labelling are shown in FIG. 2 where the lanes, from left to right, are as follows:
a) M: λPst marker
b) 1: DNA labelled with biotin 11 dCTP using Taq DNA polymerase at 37° C. for 30 minutes. Before selection.
c) 2: DNA labelled with biotin 11 dCTP using Taq DNA polymerase at 37° C. for 30 minutes. Samples taken after 3 rounds of selection.
d) 3: DNA labelled with biotin 11 dCTP using Taq DNA polymerase at 50° C. for 30 minutes. Before selection.
e) 4: DNA labelled with biotin 11 dCTP using Taq DNA polymerase at 50° C. for 30 minutes. Samples taken after 3 rounds of selection.
f) 5: Negative control PCR without DNA.

### Example 11

### Labelling with DNA Deoxynucleotidyl Transferase

The DNA template was prepared as in Example 1, the substrate was prepared as in Example 3.

Subsequently DNA was labelled by DNA terminal deoxynucleotidyl transferase in a reaction containing:
a) 17 ng model DNA,
b) 1.5 μl 1 mM Biotin 11 dCTP,
c) 10 μl 5× terminal deoxynucleotidyl transferase reaction buffer (1 M potassium cacodilate, 125 mM Tris, 0.05% Triton×100 and 5 mM CoCl₂ (pH 7.2 at 25° C.),
d) 40 U terminal deoxynucleotidyl transferase,
e) distilled H₂O to 50 μl total reaction volume.

The incubation was performed at 37° C. for 15 minutes.

DNA was selected as in Example 7 and identified as in Example 8.

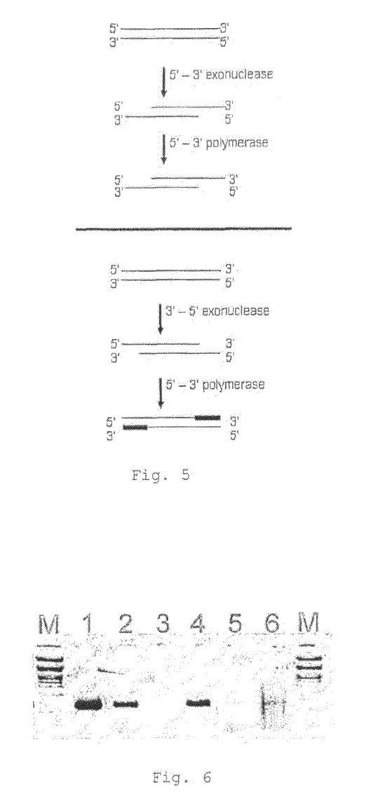

FIG. 3 shows the efficacy of DNA deoxynucleotidyl transferase in DNA labelling. This provides an alternative labelling procedure completely independent of DNA nick translation.

In FIG. 3, the lanes from left to right contain:
a) 1: Identification of terminal transferase labelled DNA before selection.
b) 2: Identification of terminal transferase labelled DNA after 3 rounds of selection.
c) 3: Negative control PCR without DNA.
d) M: λPst marker.

### Example 12

### Interference of DNA Ligase with DNA Nick Translation

In FIG. 4 we show that DNA ligase interferes with DNA nick translation in a fashion undesirable for and incompatible with SAMPAD.

The DNA template was prepared as in Example 1, the substrate was prepared as in assay 3.

T4 DNA ligase mediated DNA repair was performed on a total of 340 ng model DNA in a total reaction volume of 20 μl (2 μl 10× T4 DNA ligase reaction buffer (400 mM Tris-HCl, 100 mM MgCl₂, 100 mM DTT, 5 mM ATP, pH 7.8 at 25° C.), 5 U T4 DNA ligase and distilled H₂O to 20 μl). The incubation was performed at 37° C. for 30 minutes.

DNA was labelled as in Example 6, selected as in Example 7 and identified as in Example 8.

Nicks in the sugar phosphate backbone of DNA are repaired by DNA ligase, making it a candidate for repairing DNA nicks in vitro and so reducing the background noise of SAMPAD. However T4 DNA ligase interferes with DNA nick translation, making this potential approach to the reduction of background noise, which is obvious to one versed in the art, useless. We also observed that subtractive nick translation (meaning an initial round of labeling by nick translation and selection of DNA, before the recognition and modification of heteroduplex DNA molecules, designed to remove any pre-existing damaged DNA molecules) is of no practical use in reducing background noise.

In FIG. 4 the lanes from left to right contain:
a) M: λPst marker.
b) 1: plasmid after ligation repair, identified before selection.
c) 2: plasmid without ligation repair, identified before selection.
d) 3: plasmid after ligation repair, identified after 3 rounds of selection.
e) 4: plasmid without ligation repair, identified after 3 rounds of selection.
f) 5: plasmid after ligation repair, identified after 6 rounds of selection.
g) 6: plasmid without ligation repair, identified after 6 rounds of selection.
h) 7: plasmid after ligation repair, identified after 9 rounds of selection.
i) 8: plasmid without ligation repair, identified after 9 rounds of selection.
j) 9: plasmid after ligation repair, identified after 12 rounds of selection.
k) 10: plasmid without ligation repair, identified after 12 rounds of selection.

### Example 13

### Labeling of DNA Termini

FIG. 5 illustrates the theoretical considerations as to how DNA exonuclease and DNA polymerase activities may lead to labeling of DNA termini.

The bold line indicates the outcome of DNA polymerase activity, i.e. indicating DNA labeling by the DNA polymerase.

### Example 14

### Masking of DNA with E. coli DNA Polymerase I

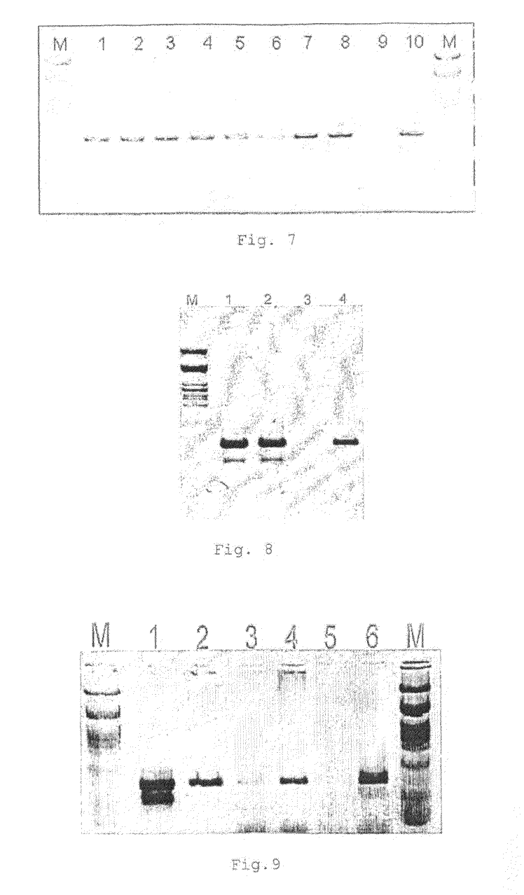

FIG. 6 shows how masking of DNA damage by E. coli DNA polymerase I using ddGTP reduces the level of background noise in a plasmid nick translation reaction.

Plasmid DNA was prepared as in Example 9 and labeled as in Example 4 with the exception that Taq DNA polymerase and Taq DNA polymerase reaction buffer were replaced by E. coli DNA polymerase I and E. coli DNA polymerase I reaction buffer respectively and the reaction was performed at 37° C.

Subsequently DNA was selected as in Example 7 and identified as in Example 8.

Two experimental conditions were compared: on the one hand DNA was masked with ddGTP and then labelled with biotin 11 dCTP and on the other hand the DNA was labelled without the masking step.

FIG. 6 clearly shows that masking drastically reduces the amount of labelling carried out.

In FIG. 6 the lanes from left to right contain:
a) M: λPst marker.
b) 1: ddGTP masking, biotin labelled, without selection.
c) 2: no ddGTP masking, biotin labelled, without selection.
d) 3: ddGTP masking, biotin labelled, 3 rounds of selection.
e) 4: no ddGTP masking, biotin labelled, 3 rounds of selection.
f) 5: negative control PCR.
g) 6: positive control PCR.
h) M: λPst marker.

### Example 15

### Labelling with E. coli DNA Polymerase I

In FIG. 7 we show that E. coli DNA polymerase I indiscriminately labels the ends of linear DNA irrespective of whether the DNA has been masked with ddGTP or not.

The DNA template was prepared as in Example 1, the substrate was prepared as in Example 3. Masking was carried out as in Example 9 (using the same quantity of DNA as used in Example 6), selection as in Example 7 and identification as in Example 8.

Here we demonstrate that E. coli DNA polymerase I labels linear DNA whether or not it has been treated with ddGTP beforehand.

We have previously shown in this document that E. coli DNA polymerase I can be used with success in masking circular DNA from subsequent E. coli DNA polymerase I labelling (FIG. 6). The substrate for DNA synthesis is produced by the 3′ to 5′ DNA exonuclease activity (see FIG. 5 for theoretical considerations).

Thus, a DNA polymerase which lacks 3′ to 5′ exonuclease activity must be used.

In FIG. 7 the lanes from left to right contain:
a) M: λPst marker
b) 1: DNA treated with ddGTP, labelled with biotin, without selection
c) 2: DNA treated with ddGTP, labelled with biotin, without selection
d) 3: DNA treated with ddGTP, labelled with biotin, without selection.
e) 4: DNA treated with ddGTP, labelled with biotin, without selection
f) 5: DNA treated with ddGTP, labelled with biotin, after 3 rounds of selection
g) 6: DNA treated with ddGTP, labelled with biotin, after 3 rounds of selection
h) 7: DNA treated with ddGTP, labelled with biotin, after 3 rounds of selection
i) 8: DNA treated with ddGTP, labelled with biotin, after 3 rounds of selection
j) 9: negative PCR control.
k) 10: positive PCR control.
l) M: λPst marker

### Assay 16

### Masking of DNA with Tag DNA Polymerase

In FIG. 8 we show that masking DNA damage and DNA termini with ddGTP using Taq DNA polymerase minimises background noise in DNA nick translation of linear DNA.

The DNA template was prepared as in Example 1, the substrate was prepared as in Example 3. Masking was carried out as in Example 4, labelled with biotin as in Example 6, selected as in Example 7 and identified as in Example 8.

In this assay two experimental conditions were compared. DNA was masked with ddGTP, labelled with biotin 11 dCTP, selected and identified and compared to DNA which had not been masked but otherwise treated identically.

Clearly, masking dramatically reduces the amount of labelling carried out.

In FIG. 8 the lanes from left to right contain:
a) M: λPst marker
b) 1: DNA masked with ddGTP, without selection
c) 2: DNA not masked with ddGTP, without selection
d) 3: DNA masked with ddGTP, 3 rounds of selection
e) 4: DNA not masked with ddGTP, 3 rounds of selection

### Example 17:

### Masking using Tag DNA Polymerase at Different Temperatures

In FIG. 9 we show that ddGTP masking using Taq DNA polymerase for incorporation functions optimally at 50° C.

The DNA template was prepared as in Example 1, the substrate was prepared as in Example 3. Masking was carried out as in Example 4 except for changes specified below, labelled with biotin as in Example 6, selected as in Example 7 and identified as in Example 8.

In this assay we show that ddGTP masking by Taq DNA polymerase performed at 60° C. is less efficient than at 50° C. In conjunction with the data presented in FIG. 2, which show Taq DNA polymerase activity to be unacceptably slow at 37° C. we take 50° C. as the optimal temperature at which to perform ddGTP masking with Taq DNA polymerase.

In FIG. 9 the lanes from left to right contain:
a) M: λPst marker.
b) 1: DNA masked at 50° C., without selection
c) 2: DNA masked at 60° C., without selection
d) 3: DNA masked at 50° C., without selection, after 3 rounds of selection
e) 4: DNA masked at 60° C., without selection, after 3 rounds of selection.
f) 5: negative control PCR.
g) 6: positive control PCR.
h) M: marcador λPst.

### Example 18

### Detection of Mismatches Processed by Mung Bean Nuclease

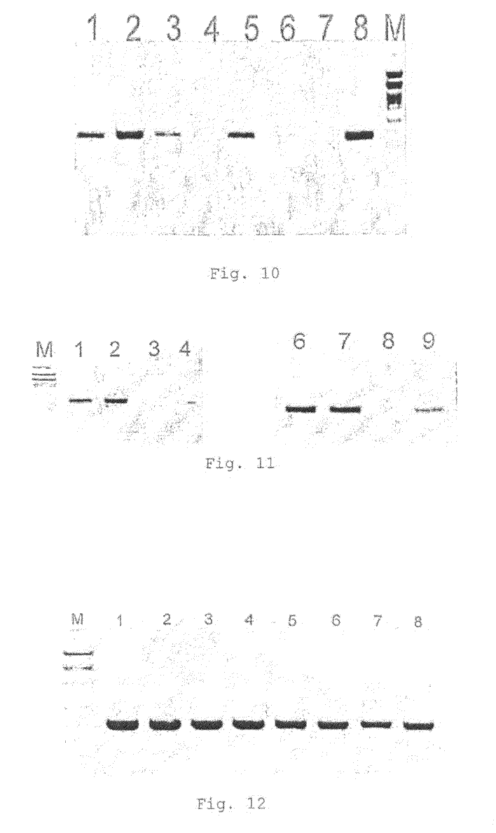

In FIG. 10 we show how mismatches processed by mung bean nuclease can be detected by SAMPAD.

DNA templates were generated as in Examples 1 and 2, the substrate DNA produced as in Example 3, masked as in Example 4 and the heteroduplex DNA molecules recognised and processed by mung bean nuclease (in brief, 8.5 ng of masked DNA were incubated in a total reaction volume of 20 μl with 2 μl 10× mung bean nuclease reaction buffer (300 mM sodium acetate (pH 4.6), 500 mM NaCl, 10 mM Zn acetate and 0.1% Triton×100), 50 U Mung bean nuclease at 37° C. for 15 minutes) and labelled with biotin directly from the nuclease reaction. DNA was labelled as in Example 6, was selected as in Example 7, and was identified as in Example 8.

Nuclease treated DNA was labelled with biotin without purification.

In FIG. 10 the lanes from left to right contain:
a) 1: Matched DNA, treated with mung bean nuclease, without selection.
b) 2: mismatched DNA, treated with mung bean nuclease, without selection.
c) 3: control for nick translation efficiency, without selection.
d) 4: Matched DNA, treated with mung bean nuclease, without selection, 3 rounds of selection.
e) 5: Mismatched DNA, treated with mung bean nuclease, without selection, 3 rounds of selection.
f) 6: control for nick translation efficiency, 3 rounds of selection.
g) 7: negative control PCR.
h) 8: positive control PCR.
i) M: λPst marker.

### Example 19

### Recognition and Modification with SURVEYOR™ Nuclease

In FIG. 11 we show that low levels of SURVEYOR™ nuclease for mismatch recognition and modification and short incubation times (2 to 7 minutes) permit efficient trapping of nicked DNA (processed heteroduplex DNA).

DNA templates were prepared as in Examples 1 and 2, the substrate prepared as in Example 3, masking performed as in Example 4, heteroduplex molecules identified as in Example 5 (with the exception that the amount of SURVEYOR™ enzyme per reaction was varied), it was purified as is shown in Example 20, labelled as in Example 6, selected as in Example 7 and identified as in Example 8.

The SURVEYOR™ nuclease preparation and recommended application conditions for routine mutation detection (1 μl SURVEYOR™ nuclease per reaction; incubation time 20 minutes at 42° C.) are designed to maximise the efficiency of mismatch cutting (i.e. cleavage of both strands).

Here we demonstrate that shorter incubation times and reduced enzyme concentrations (0.1 μl μl SURVEYOR™ nuclease per reaction, 5 minutes incubation at 42° C.) resulted in efficient production of nicks (which can be labelled efficiently by DNA nick translation).

It is also shown that using half the amount of nuclease employed in the standard TRANSGENOMIC mismatch cutting reaction (i.e. 0.5 l μl SURVEYOR™ nuclease per reaction; incubation time 5 minutes at 42° C.) does not permit trapping of nicked DNA molecules. This is probably a case of over digestion, molecules being comprehensively cut rather than nicked.

DNA was purified using spin columns after SURVEYOR™ nuclease treatment.

In FIG. 11 the lanes from left to right contain:
a) M: λPst marker
b) 1: 100% matched DNA, 0% mismatched DNA, 0.5 μl SURVEYOR™, without selection
c) 2: 50% matched DNA, 50% mismatched DNA, 0.5 μl SURVEYOR™, without selection
d) 3: 100% matched DNA, 0% mismatched DNA, 0.5 μl SURVEYOR™, 3 rounds of selection
e) 4: 50% matched DNA, 50% mismatched DNA, 0.5 μl SURVEYOR™, 3 rounds of selection
f) 5: negative PCR control
g) 6: 100% matched DNA, 0% mismatched DNA, 0.1 μl SURVEYOR™, without selection
h) 7: 50% matched DNA, 50% mismatched DNA, 0.1 μl SURVEYOR™, without selection
i) 8: 100% matched DNA, 0% mismatched DNA, 0.1 μl SURVEYOR™, 3 rounds of selection
j) 9: 50% matched DNA, 50% mismatched DNA, 0.5 μl SURVEYOR™, 3 rounds of selection

### Example 20

### Effect of Standard Ethanol Precipitation of DNA on Masking

Templates were prepared as in Example 1, substrates generated as in Example 3, masked as in Example 4. Masked DNA was precipitated by standard ethanol precipitation. DNA was labelled as in Example 6, selected as in Example 7 and identified as in Example 8.

SURVEYOR™ nuclease reaction conditions completely inhibit DNA polymerase activity, thus inhibiting the labelling reaction.

Mung Bean Nuclease reaction conditions severely impair Taq DNA polymerase, thus precluding efficient labelling. To circumvent this problem we investigated ways to change reaction conditions and so to obtain a fully functional assay.

Here we test whether standard ethanol precipitation of DNA following the masking reaction is suitable. It is not suitable. We find ethanol precipitation inflicts so much damage on the DNA as to completely negate the benefits of the masking reaction.

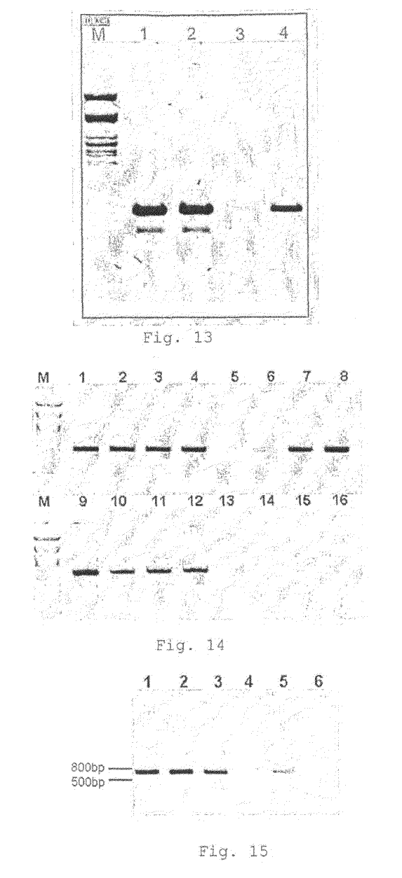

In FIG. 12 the lanes from left to right contain:
a) M: λPst marker
b) 1,2: DNA masked with ddGTP and precipitated with ethanol before labelling, without selection
c) 3,4: DNA not masked with ddGTP and precipitated with ethanol before labelling, without selection
d) 5,6: DNA masked with ddGTP and precipitated with ethanol before labelling, 3 rounds of selection
e) 7,8: DNA not masked with ddGTP and precipitated with ethanol before labelling, 3 rounds of selection

### Example 21

### Effect of Spin Column Purification on Masking

To investigate the possibility of a more gentle purification procedure, DNA templates were prepared as in Example 2, substrate generated as in Example 3, masked as in Example 4, purified using Millipore montage centrifugation columns (using manufacturers recommendations), labelled as in Example 6, selected as in Example 7 and identified as in Example 8.

In FIG. 13 we show that spin column purification provokes little increase in background labelling. Thus spin column purification is a DNA purification method sufficiently gentle for SAMPAD and is now routinely employed.

In FIG. 13 the lanes from left to right contain:
a) M: λPst marker
b) 1: DNA masked with ddGTP, purified by spin column purification, without selection.
c) 2: DNA not masked with ddGTP, purified by spin column purification, without selection.
d) 3: DNA masked with ddGTP, purified by spin column purification, 3 rounds of selection.
e) 4: DNA not masked with ddGTP, purified by spin column purification, 3 rounds of selection.

### Example 22

### Entire SAMPAD Process

In FIG. 14 we show how, taking into account parameters determined in previous assays, mutations can efficiently be detected with SAMPAD.

DNA templates were prepared as in Examples 1 and 2, substrates prepared as in Example 3, masked as in Example 4, heteroduplex structures recognised and modified as in Example 5, purified as in Example 20, labelled as in Example 6, selected as in Example 7 and identified as in Example 8.

Here we show that SAMPAD is sufficiently sensitive to identify one mutant in the presence of 255 normal molecules.

In FIG. 14 the lanes from left to right contain:
a) M: λPst marker
b) 1: 100% matched DNA, 0% mismatched DNA, without selection
c) 2: 99.2% matched DNA, 0.8% mismatched DNA, without selection
d) 3: 99.6% matched DNA, 0.4% mismatched DNA, without selection
e) 4: control for the labelling reaction, without selection
f) 5: 100% matched DNA, 0% mismatched DNA, without selection, 3 rounds of selection
g) 6: 99.2% matched DNA, 0.8% mismatched DNA, without selection, 3 rounds of selection
h) 7: 99.6% matched DNA, 0.4% mismatched DNA, 3 rounds of selection
i) 8: control for the labelling reaction, 3 rounds of selection
j) 9: internal control to number 1.
k) 10: internal control to number 2.
l) 11: internal control to number 3.
m) 12: internal control to number 4.
n) 13: internal control to number 5.
o) 14: internal control to number 6.
p) 15: internal control to number 7.
q) 16: internal control to number 8.

### Example 23

### Purification and Sequencing of the SAMPAD Product

The product of SAMPAD was purified and sequenced directly (without cloning of the PCR products). DNA templates were prepared as in Examples 1 and 2, substrates prepared as in Example 3, masked as in Example 4, heteroduplex structures recognised and modified as in Example 5, purified as in Example 20, labelled as in Example 6, selected as in Example 7 and identified as in Example 8 with the exception that specific primers described in Example 1 were used.

Detection products were directly sequenced with one of the amplification primers (Example 1) using the Applied Biosystems BigDye kit.

This possibility is a clear advantage over the method of Makrigiorgos [Makrigiorgos, Gerrasimos M., Methods for rapid screening of polymorphisms, mutations and methylation, US patent application US20030022215], which permits amplification of the fragment immediately flanking the mutation but where the direct information about the mutation is lost as it is not present in the end product of the procedure. This also improves on the method by Yeung, which provides positional information regarding the mutation (see claims 7 and 8 of U.S. patent application PCT/US97/08705), but does not reveal its nature.

### Example 24

### Identification of Variations Between Different Strains of the Same Yeast Species

In a second industrial application of the invention disclosed here we can identify variations between different strains of the same yeast species.

Genomic DNA was prepared from yeast strain A and yeast strain B, both of which belong to the same species, Saccharomyces cerevisae.

DNA was digested with restriction enzymes SacI (Fermentas) and MseI (New England Biolabs). 1 μg DNA was digested with 15 units SacI in SacI+ buffer (Fermentas) in a total volume of 40 μl. This was further supplemented with 4 μl NEB2 buffer and 0.5 μl 10 mg/ml BSA and 15 units MseI for the second digestion.

Once digested, synthetic adaptors were ligated using T4 DNA ligase under standard ligation reaction conditions:
SacI adaptor (SEQ ID No.9 and SEQ ID No.10)
MseI adaptor (SEQ ID No.11 and SEQ ID. No.12)

The SacI adaptor was labelled with biotin.

Subsequently biotin labelled genome fragments were selected as in Example 7.

Now selected DNA was amplified as in Example 8, but using primers identified as SEQ ID No.13 and SEQ ID. No.14 and Pfu DNA polymerase.

Products from the amplification of strain 1 and a mixture of the products amplified from strains 1 and 2 were added to substrates produced as in Example 3, making substrates 1 (matched) and 2 (mismatched)

From this material differences between strains were determined.

Substrate 1 permits us to determine whether the presence of complex DNA mixtures raises the level of background noise to an unacceptable level. Substrate 2 allows us to investigate if SAMPAD works in the presence of a complex DNA mixture.

SAMPAD was performed as in Example 20. As is shown in FIG. 16, complex DNA does not interfere with the correct functioning of SAMPAD.

To determine if heteroduplex molecules can be identified from within a complex mixture of DNA, magnetic particles were resuspended in distilled H₂O and 1 μl used as a substrate for PCR amplification as in Example 3 with the exception that biotin labelled primers were used. This generated PCR product 1 (derived from strain 1) and PCR product 2 (derived from a mixture of the two strains).

PCR products 1 and 2 were precipitated as in assay 20 and resuspended in DNA hybridisation buffer (1XMES, 200 μl/ml Herring sperm DNA, 1 mg/ml bovine serum albumin) and incubated at 95° C. for 10 minutes, yielding the ready to use hybridisation mixture.

Hybridisation mixtures were hybridised to DNA chips for 12 hours. Chips bore oligonucleotides capable of recognising the various genomic SacI restriction sites.

Comparison of the fragments present in the sample derived from strain 1 versus the sample derived from the mixture of the two strains highlights the sequence difference between the two strains.

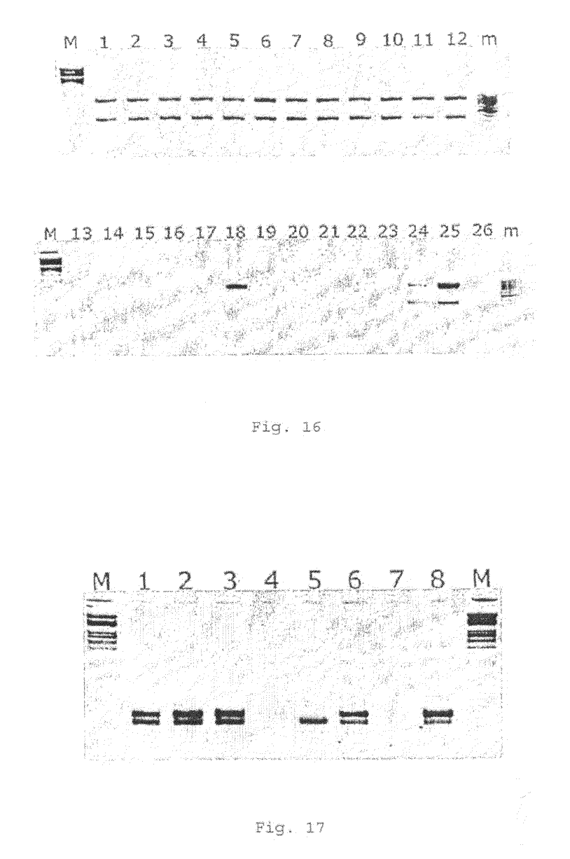

In FIG. 15 the lanes from left to right contain:
a) M: marker λPst
b) 1: 100% matched DNA, 0% mismatched DNA, selection with strain 1 yeast DNA, before selection
c) 2: 50% matched DNA, 50% mismatched DNA, selection with mixed strain 1 and strain 2 yeast DNA, before selection.
d) 3: positive control for DNA nick translation, before selection.
e) 4: 100% matched DNA, 0% mismatched DNA selection with strain 1 yeast DNA, 3 rounds of selection.
f) 5: 50% matched DNA, 50% mismatched DNA selection with mixed strain 1 and strain 2 yeast DNA, after 3 rounds of selection.
g) 6: positive contol for DNA nick translation, after 3 rounds of selection

### Example 25

### SAMPAD Screening Experiment

In order to test the robustness of the method of the present invention in screening for rare mutations a blind experiment was performed. This experiment consisted of the detection of mismatched DNA molecules in one of 10 samples without the operator knowing which sample contains the mismatched DNA. This situation mimicks the real application of this method where rare mutations are detected in an overwhelmingly wild type background. Ten separate reactions were performed only one of which contained a variant sequence in a ratio 1:128, i.e. 1 variant DNA molecule per 128 molecules.

Also, in this experiment an internal control DNA was added to validate the reproducibility of the assay. This internal control harbours no variability and is put through all steps of the reaction together with the DNA in which variation is being sought.

DNA templates were prepared as in Examples 1 and 2 and substrates prepared as in Example 3. Internal control template was prepared analogous to Example 1 with the exception that the amplification product of primers Fut.F (SEQ ID No.17) and Fut.R (SEQ ID No.18) on rice genomic DNA was digested with appropriate restriction enzymes and inserted into an appropriately digested plasmid. Internal control substrate was prepared analogous to Example 3 but employing primers Fut.F (SEQ ID No.17) and Fut.R (SEQ ID No.18).

Masking was performed as in Example 4, heteroduplex structures recognised and modified as in Example 5, purified as in Example 20, labelled as in Example 6, selected as in Example 7 and identified as in Example 8 with the exception that primers CaEnh (SEQ ID No.15) and GFPR (SEQ ID No.16) were used for the sample DNA, yielding a 900 bp fragment, and Fut.F (SEQ ID No.17) and Fut.R (SEQ ID No.18) were used for the internal control DNA, yielding a 320 bp fragment.

Thus, as shown in FIG. 16, this experiment indicates that the method of the present invention is sufficiently sensitive to efficiently identify one mutant molecule per 128 molecules.

In FIG. 16, the lanes from left to right contain:
M: λPst marker
m: 100 bp ladder
1: matched substrate, matched internal masking control, before selection
2: matched substrate, matched internal masking control, before selection
3: matched substrate, matched internal masking control, before selection
4: matched substrate, matched internal masking control, before selection
5: matched substrate, matched internal masking control, before selection
6: 1/128 mismatched substrate, matched internal masking control, before selection
7: matched substrate, matched internal masking control, before selection
8: matched substrate, matched internal masking control, before selection
9: matched substrate, matched internal masking control, before selection
10: matched substrate, matched internal masking control, before selection
11: matched substrate, matched internal masking control, before selection
12: labelling control, before selection
13: matched substrate, matched internal masking control, 9 rounds of selection
14: matched substrate, matched internal masking control, 9 rounds of selection
15: matched substrate, matched internal masking control, 9 rounds of selection
16: matched substrate, matched internal masking control, 9 rounds of selection
17: matched substrate, matched internal masking control, 9 rounds of selection
18: 1/128 mismatched substrate, matched internal masking control, 9 rounds of selection
19: matched substrate, matched internal masking control, 9 rounds of selection
20: matched substrate, matched internal masking control, 9 rounds of selection
21: matched substrate, matched internal masking control, 9 rounds of selection
22: matched substrate, matched internal masking control, 9 rounds of selection
23: matched substrate, matched internal masking control, 9 rounds of selection
24: labelling control, 9 rounds of selection
25: positive PCR control
26: negative PCR control

### Example 26

### Detection of Mutations in the APC Gene

In this example mutations in the human adenomatous polyposis coli (APC) gene were analysed. These mutations are frequently associated with the development of colon cancers in humans. Mutations are most frequently encountered in the mutation cluster region (MCR) of exon 15 of the APC gene. There are however no characteristic specific sites (“hot spots”) where mutations are typically encountered; rather the MCR in its entirety is a “hot region”, a wide range of mutations frequently encountered in this region.

This lack of characteristic specific mutations precludes the application of methodologies geared to detection of specific mutations as only a minor, for a diagnostic assay unacceptably low, subset of causative mutations would be pinpointed. The APC MCR is a suitable target for the method of the present invention.

As observed in FIG. 17, in this example we show that the method of the present invention allows the detection of a frameshift mutation in the APC MCR in a patient heterozygous for such a mutation.

### Experimental Outline

Amplification of genomic DNA from exon 13 including codons 1239 to 1561 of exon 15 of the APC gene (SEQ ID No.22) from patient samples was performed with Pfu polymerase using primers APCl (SEQ ID No.19) and APC2 (SEQ ID No.20) under standard reaction and thermal cycling conditions (for example, as described in Example 3).

As in Example 25, an internal control DNA was added to validate the reproducibility of the assay. Primers used for the amplification of this control DNA were Fut.F (SEQ ID No.17) and Fut.R (SEQ ID No.18).

Masking was performed as in Example 4, heteroduplex structures recognised and modified as in Example 5, purified as in Example 20, labelled as in Example 6 and selected as in Example 7. Identification by PCR analysis was performed as in Example 8 with primer pair APC2 (SEQ ID No.20) and APC3 (SEQ ID No.21). This amplifies a portion of the APCR MCR analysed for mutations in this assay.

Amplification of diagnostic APC yielded a product of 287 bp (SEQ ID No.23) and the non-mutated control DNA yielded a product of 357 bp (SEQ ID No.24).

In FIG. 17, the lanes from left to right contain:
M: λPst marker
1: APC DNA without frameshift mutation, non-mutated reference sample, before selection
2: APC DNA with frameshift mutation, non-mutated reference sample, before selection
3: labelling control reaction, APC DNA and non-mutated reference sample, before selection
4: APC DNA without frameshift mutation, non-mutated reference sample, 6 rounds of selection
5: APC DNA with frameshift mutation, non-mutated reference sample, 6 rounds of selection
6: labelling control reaction, APC DNA and non-mutated reference sample, 6 rounds of selection
7: negative PCR control
8: positive PCR control

1. A process for selectively detecting nucleic acid molecules comprising structural features that are capable of being converted into nicks comprising:
a) generating linear nucleic acids from a selected nucleic acid substrate population;
b) de-naturing and re-annealing the linear nucleic acids to form nucleic acid duplexes;
c) masking the nucleic acid duplex termini and internal structural features with a masking component;
d) modifying the masked nucleic acids by introducing nicks therein using at least an enzyme possessing endonuclease activity;
e) labelling the modified nucleic acids with labelled nucleotides via nucleic acid nick translation with at least an enzyme displaying a nucleic acid polymerase activity; and
f) selecting and identifying the labelled nucleic acid.

2. A process according to claim 1 wherein the nucleic acid substrate population is obtained from a natural or a synthetic source.

3. A process according to claim 1 wherein the nucleic acid substrate population is obtained from a source selected from the group consisting of a eukaryote, a prokaryote, a synthetic nucleic acid, plasmidic, a synthesized vector, a peptidic nucleic acid, an RNA virus, and a DNA virus sample.

4. A process according to claim 1, wherein the masking component is at least an enzyme that has 5′-3′ DNA polymerase activity and terminal deoxynucleotidyl transferase activity and lacks 3′-5′ exonuclease activity and is capable of catalysing the addition of a nucleotide component such as a nucleotide or a nucleotide analogue to nucleic acid duplex termini and internal structural features of the nucleic acid duplex that are recognisable by the enzyme.

5. A process according to claim 4 wherein the nucleotide component is a dideoxynucleotide, such as dideoxyguanosine triphosphate (ddGTP).

6. A process according to claim 4 wherein the incorporation of protective groups which are nucleotides or nucleotide analogues is carried out by more than one enzyme having DNA polymerase enzyme activity and lacking 3′-5′ exonuclease activity.

7. A process according to claim 4 wherein the enzyme is a DNA polymerase enzyme or a Taq DNA polymerase.

8. A process according to claim 7 wherein the masking step is performed at a temperature in the range selected from the group consisting of from 37° C. to 60° C., from 45° C. to 55° C., and from 48° C. to 52° C.

9. A process according to claim 7 wherein the masking step is performed for a time interval selected from the group consisting of between about 30 minutes and 18 hours, between 45 minutes and 10 hours, and between about 60 minutes and 120 minutes.

10. A process according to claim 1, wherein the recognising and modifying of masked nucleic acids is performed for a short incubation period using at least a mismatch endonuclease enzyme.

11. A process according to claim 10 wherein the mismatch endonuclease enzyme is selected from the group consisting of Cell mismatch nucleases, Cell “SURVEYOR”, single strand specific endonucleases, S1 nuclease, and mung bean nuclease.

12. A process according to claim 10, wherein the incubation period lies between about 2 to 7 minutes.

13. A process according to claim 10, wherein the incubating temperature lies in the range of from about 37° C. to 45° C.

14. A process according to claim 1, wherein recognition and modification of heteroduplex NA molecules is performed via a chemical reaction.

15. A process according to claim 1, wherein labelling of modified nucleic acids is done with an enzyme selected from the group consisting of a DNA polymerase, a Taq polymerase, a DNA terminal deoxynucleotidyl transferase, and any combination thereof.

16. A process according to claim 15 wherein the nucleotide label is biotin.

17. A process according to claim 1, wherein the selection of labelled nucleic acids is performed using streptavidin coated magnetic particles.

18. A process according to claim 17 wherein labelled nucleic acids are identified using PCR.

19. A process according to claim 1, wherein the substrate population consists of RNA.

20. A process according to claim 1, wherein the substrate population consists of DNA.

21. A process according to claim 1, wherein the nucleic acid substrate population consists of peptide nucleic acid molecules having a non-native structure and that are capable of interacting with naturally occurring nucleic acid molecules.

22. A process according to claim 1, wherein the nucleic acid substrate population is any mixture of eukaryotic, prokaryotic, viral and plasmidic nucleic acids.

23. A process according to claim 1, wherein the substrate nucleic acid molecules are obtained by direct extraction or are obtained by in vitro amplification or are obtained synthetically.

24. A kit comprising:
means for preparing a population of linearised nucleic acid molecule(s);
at least a masking agent;
at least a masking component;
at least a nicking enzyme comprising endonuclease activity;
at least a labelling agent; and
at least an enzyme preparation comprising or displaying nucleic acid polymerase activity.

25. A kit according to claim 24 further comprising:
a masking agent selected from the group consisting of dideoxynucleotides, ddGTP, other nucleotide analogues, and AZT.

26. A kit according to claim 25 further comprising an enzymatic masking component or a chemical masking preparation.

27. A kit according to claim 25 which comprises an enzymatic masking component that is at least an enzyme that has 5′-3′ DNA polymerase activity and has terminal deoxynucleotidyl transferase activity and lacks 3′-5′ exonuclease activity and is capable of catalysing the addition of a nucleotide component and internal structural features of the nucleic acid duplex that are recognisable by the enzyme.

28. A kit according to claim 27 comprising at least a nicking enzyme comprising endonuclease activity.

29. A kit according to claim 24, further comprising biotin as a labelling agent.

30. A kit according to claim 24, further comprising an enzyme preparation displaying nucleic acid polymerase activity wherein the enzyme preparation is selected from DNA polymerase, Taq polymerase and/or a DNA terminal deoxynucleotidyl transferase.


SEQUENCE LISTING (24 sequences)

SEQ ID NO: 1 - DNA, 29 bp, Artificial Sequence
  wild type template
catgtcgcgcgcgcgatatatggggggta

SEQ ID NO: 2 - DNA, 29 bp, Artificial Sequence
  wild type template
catgtaccccccatatatcgcgcgcgcga

SEQ ID NO: 3 - DNA, 31 bp, Artificial Sequence
  mutant template
catgtcgcgcgcgcgatatattaggggggta

SEQ ID NO: 4 - DNA, 31 bp, Artificial Sequence
  mutant template
catgtacccccctaatatatcgcgcgcgcga

SEQ ID NO: 5 - DNA, 17 bp, Artificial Sequence
  M13 sequencing primers
gtaaaacgacggccagt

SEQ ID NO: 6 - DNA, 17 bp, Artificial Sequence
  M13 sequencing primers
caggaaacagctatgac

SEQ ID NO: 7 - DNA, 18 bp, Artificial Sequence
  Identification primer
gtaaaggagaagaacttt

SEQ ID NO: 8 - DNA, 20 bp, Artificial Sequence
  Identification primer
ttatttgtatagttcatcca

SEQ ID NO: 9 - DNA, 22 bp, Artificial Sequence
  Sac I adaptor oligonucleotide
ctcgtagactgcgtacaagctc

SEQ ID NO: 10 - DNA, 14 bp, Artificial Sequence
  Sac I adaptor oligonucleotide
tgtacgcagtctac

SEQ ID NO: 11 - DNA, 19 bp, Artificial Sequence
  Mse I adaptor oligonucleotide
gacgatgagtcctgagtaa

SEQ ID NO: 12 - DNA, 14 bp, Artificial Sequence
  Mse I adaptor oligonucleotide
tactcaggactcat

SEQ ID NO: 13 - DNA, 22 bp, Artificial Sequence
  Yeast identification primer
ctcgtagactgcgtacaagctc

SEQ ID NO: 14 - DNA, 19 bp, Artificial Sequence
  Yeast identification primer
gacgatgagtcctgagtaa

SEQ ID NO: 15 - DNA, 20 bp, Artificial Sequence
  CaEnh primer
cgaatctcaagcaatcaagc

SEQ ID NO: 16 - DNA, 30 bp, Artificial Sequence
  GFP.R primer
cgatgatcacttatttgtatagttcatcca

SEQ ID NO: 17 - DNA, 26 bp, Artificial Sequence
  Fut.F primer
ggatccctctttcgggaaatgcttga

SEQ ID NO: 18 - DNA, 28 bp, Artificial Sequence
  Fut.R primer
cgaagctttaggctacgggatcatttgc

SEQ ID NO: 19 - DNA, 20 bp, Artificial Sequence
  APC1 primer
gaggcagaatcagctccatc

SEQ ID NO: 20 - DNA, 20 bp, Artificial Sequence
  APC2 primer
cacaatacacccgtggcata

SEQ ID NO: 21 - DNA, 22 bp, Artificial Sequence
  APC3 primer
ccaagagaaagaggcagaaaaa

SEQ ID NO: 22 - DNA, 1253 bp, Homo sapiens
gaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtg

SEQ ID NO: 23 - DNA, 287 bp, Homo sapiens
ccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtg

SEQ ID NO: 24 - DNA, 357 bp, Oryza sp.
ggatccctctttcgggaaatgcttgaataatgttgatggtcctgacatggcattatccatgtatccagtttgttcaaccaatgataatggaaaaccacactggtgggatcatcttcactgcgcaatgtcacattacaagtttgttcttgcaattgagaatactaagacagaaagctatgtgaccgagaaattgttctatgccttggaggctgggtcagtgccaatatacttcggggcacccaatgtctgggacttcattcctcccaattctattatagatgcctcaaaattcagctcactccgtgagttggcatcatatgtgaaagctgttgcaaatgatcccgtagcctaaagctt